(19) United States
(12) Plant Patent Application Publication
(10) Pub. No.: US 2020/0113105 P1
(43) Pub. Date: Apr. 9, 2020

(54) Calla lily plant named 'Night Club'

(50) ;

(71) Applicant: Dümmen Group B.V., De Lier (NL)

(72) Inventor: Peter Beckman, Santa Cruz, CA (US)

(21) Appl. No.: 16/602,421

(22) Filed: Oct. 1, 2019

Related U.S. Application Data
(60) Provisional application No. 62/766,205, filed on Oct. 4, 2018.

(51) Int. Cl. A01H 6/56

(52) U.S. Cl. PLT/490

(57) ABSTRACT

‘Night Club’ is a new variety of calla lily plant having purple colored spathes that have a trumpet shape. The flower stems are strong and long lasting. The leaves are green with no maculations present.

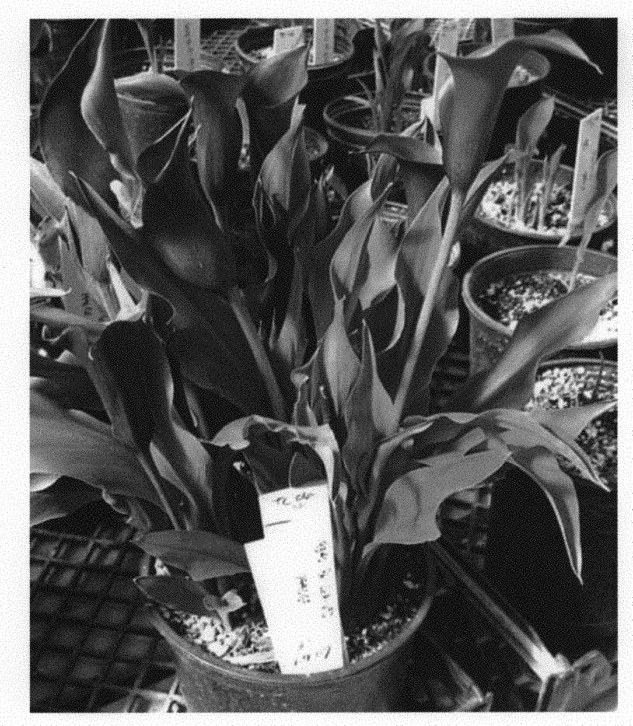

### CROSS REFERENCE TO RELATED APPLICATION

This application claims the benefit of U.S. Provisional Patent Application Ser. No. 62/766,205, having a filing date of Oct. 4, 2018, and entitled “Varieties of Z. sprengeri Calla Lily Plants”, the entire disclosure of which is incorporated herein by reference.

Botanical classification: Zantedeschia sprengeri.

Varietal denomination: ‘Night Club’.

### BACKGROUND OF THE NEW PLANT

The present invention comprises a new and distinct cultivar of calla lily, botanically known as Zantedeschia sprengeri and hereinafter referred to by the cultivar name ‘Night Club’.

‘Night Club’ is a product of a planned breeding program and the new cultivar was selected from the crossing of Zantedeschia seedling 19300 (female parent, unpatented) with Zantedeschia seedling 19300 (male parent, unpatented). ‘Night Club’ was selected in Santa Cruz, Calif. as one flowering plant within the progeny of the stated cross.

Asexual reproductions of ‘Night Club’ have demonstrated that the combination of characteristics as herein disclosed for the new variety are retained and reproduced true to type through successive generations of asexual reproduction.

Color references are made to The 2001 R.H.S. Colour Chart of The Royal Horticultural Society of London, 4^{th} Edition, except where general color terms of ordinary significance are used.

The present invention has not been evaluated under all possible environmental conditions. The phenotype may vary with variations in environment without a change in the genotype of the plant.

The following traits have been repeatedly observed and determined to be basic characteristics of ‘Night Club’ which, in combination, distinguish this calla lily as a new and distinct cultivar:

1. Purple colored spathes;

2. Trumpet shaped spathes; and

3. Firm, long-lasting peduncles.

Table 1 provides a comparison of ‘Night Club’ to similar Zantedeschia sprengeri varieties ‘Night Life’ (unpatented) and ‘Paco’ (U.S. Plant Pat. No. 24,246).

TABLE 1

| Comparison | Differing | Comparison | Present variety |
|---|---|---|---|
| variety | trait(s) | expression | expression |
| ‘Night Life’ | Spathe color | Red-Purple | Purple Group |
| | | Group RHS 59A | RHS 79A |
| | Throat spot | Present | Absent |
| ‘Paco’ | Flowering | 70-75 days to | 40-45 days to |
| | time | flower | flower |

Further, ‘Night Club’ has more trumpet shaped, smoother edged, and narrower inflorescences than ‘Night Life’. ‘Night Club’ flowers 7 days earlier than ‘Night Life’. Also, ‘Night Club’ exhibits superior peduncle strength (21 days to nodding) than ‘Night Life’ (12 days to nodding).

### BRIEF DESCRIPTION OF THE DRAWING

The accompanying photographic drawing illustrates the new variety, with the colors being as nearly true as is possible with color illustrations of this type.

### DESCRIPTION OF THE NEW PLANT

Type: Deciduous.
Main use: Potted plant.
Flowering time: 40-45 days to flower.
Disease/pest resistance: Strong tolerance to Pythium root rot and Erwinia bulb soft rot.
Leaves: Green, with no maculations present.
Spathe:
   Shape.—Trumpet.
   Length.—Short to medium.
   Width.—Narrow to medium.
   Color.—Throat spot: Absent. Upper surface: Purple Group RHS 79A.
Peduncle: Strong and erect, with no nodding observed until day 21.

I claim:

1. A new and distinct variety of calla lily plant named ‘Night Club’ as herein described and illustrated.
